(19) United States
(12) Patent Application Publication
(10) Pub. No.: US 2022/0156573 A1
(43) Pub. Date: May 19, 2022

(54) Machine Learning Engine Providing Trained Request Approval Decisions

(71) Applicant: TOTVS INC (DBA TOTVS Labs), Raleigh, NC (US)

(72) Inventors: Rafael Rui, Rio Grande do Sul (BR); Scott Brownlie, Rio de Janeiro (BR); Renan Alves Fonseca, Florianópolis (BR); Mithun Puthige Acharya, Cary, NC (US); Vincent Goetten, Raleigh, NC (US)

(21) Appl. No.: 16/951,110

(22) Filed: Nov. 18, 2020

(51) Int. Cl. G06N 3/08; G06K 9/62; G06F 9/30

(52) U.S. Cl. CPC G06N 3/08; G06F 9/3001; G06F 9/30036; G06K 9/6259

(57) ABSTRACT

Systems, devices, and methods for automated approval of claim requests for solicited procedures. In an embodiment, a system includes an audit manager and an attention-based neural network. A computer-readable memory stores tuning parameters and a set of risk level thresholds. A database is configured to store training data including fixed length and variable length data. Fixed length data includes features and a target label. Variable length data includes medical procedure code approval history data. Validation data and operation data may also be stored in the database. The audit manager is configured to output an approval indication and rejection probability score for each solicited procedure according to a selected risk level threshold in the set of risk level thresholds. In one feature, an attention-based neural network is trained according to features and target label in the fixed length data and medical procedure code approval history data in the variable length data.

### TECHNICAL FIELD

The technical field of the present disclosure relates to computer-implemented machine learning in approval and audit decisions.

### BACKGROUND ART

In many industries, solicited procedures are evaluated to determine whether to approve the solicited procedures. One conventional approach relies upon human experts to evaluate each solicited procedure and manually assess whether to approve or disapprove of a solicited procedure. This can be cost-prohibitive and time consuming and not able to scale to handle large volumes of solicited procedures quickly.

Machine learning techniques are increasingly sought to automate aspects of decision making. See, R. Burri et al., “Insurance Claim Analysis Using Machine Learning Algorithms,” Int'l Jn. Of Innovative Tech. and Exploring Engineering (IJITEE), Vol. 8, Issue SS4, April 2019, pp. 577-582. However, machine learning often involves complex feature engineering or is limited to fixed length data with simple relationships known a priori between tables of data in a relational database.

For example, prior supervised machine learning models such as logistic regression, support vector machines and random forests are fit to a set of training examples, where each example is a pair consisting of a fixed length feature vector and its associated fixed length label. When working with relational databases it is rare that useful feature vectors are readily available in a single table. More commonly, they must be carefully constructed from several tables in a process which is known as feature engineering. This process, which often requires domain-specific knowledge and accounts for the vast majority of man hours on a data science project, can involve multiple joins, filters, groupings and aggregations.

One particular challenge when constructing features from relational databases is deciding how to resolve one-to-many relationships. Consider, for example, the task of predicting whether customers of a retail website will churn. The purchase history of any given customer will almost certainly be useful for this task, but it is difficult to assess how one presents this information to a neural network when multiple orders for most customers and the number of orders can vary significantly for different customers. This difficulty is often compounded by a lack of domain-specific expert knowledge regarding a data set. It is not uncommon for data scientists to spend a considerable amount of time constructing every possible feature they can think of in a trial and error manner, only to later discover that many of them are completely useless for the prediction task. Moreover, when there are many tables in the database the number of possibilities for feature engineering can seem endless, overwhelming, and cost-prohibitive.

One attempt to automate feature engineering from relational databases is with a rule-based approach where transformations to be applied to data are specified a-priori by a user. See, e.g., James Max Kanter and Kalyan Veeramachaneni, “Deep feature synthesis: Towards automating data science endeavors,” 2015 IEEE International Conference on Data Science and Advanced Analytics (DSAA), Volume 113, IEEE, October 2015, pages 1-10; Randal S. Olson, Nathan Bartley, Ryan J. Urbanowicz, and Jason H. Moore, “Evaluation of a Tree-based Pipeline Optimization Tool for Automating Data Science,” Proceedings of the 2016 on Genetic and Evolutionary Computation Conference—GECCO '16, ACM Press, New York, N.Y., USA, 2016, pp. 485-492; and Gilad Katz, Eui Chul Richard Shin, and Dawn Song, “ExploreKit: Automatic Feature Generation and Selection,” 2016 IEEE 16th International Conference on Data Mining (ICDM), IEEE, December 2016, pp. 979-984. Much like a human would do, these methods generate a large number of problem independent features, many of which are irrelevant and later eliminated in a feature selection step.

Deep learning models, which have become extremely popular in recent years, have the ability to automatically learn useful features directly from a training error signal. In computer vision, most state-of-the-art results are now achieved by convolutional neural networks (CNNs) which learn to extract rich, hierarchical features from the raw image pixels. In natural language processing (NLP), recurrent neural networks (RNNs) can learn from variable-length sequences of words, making them more flexible than conventional models.

Recent works on automated feature engineering for relational databases also use RNNs to learn useful feature representations from labelled data rather than the transformations being specified a priori by the user. See, e.g., Hoang Thanh Lam, Tran Ngoc Minh, Mathieu Sinn, Beat Buesser, and Martin Wistuba, “Neural Feature Learning From Relational Database,” arXiv:1801.05372v4, 15 Jun. 2019, pp. 1-15; J Moore and J Neville, “Deep collective inference,” 31st AAAI Conference on Artificial Intelligence, AAAI 2017, number 1, 2017, pp. 2364-2372; and Manzil Zaheer, Satwik Kottur, Siamak Ravanbakhsh, Barnabas Poczos, Ruslan Salakhutdinov, and Alexander Smola, “Deep Sets,” Advances in Neural Information Processing Systems, 31 Conf. on Neural Information Processing Systems (NIPS 2017), Long Beach, Calif., 11 pages. However, the time required to train several RNNs is a major hindrance and makes their use infeasible for the majority of data scientists without access to significant computational resources.

What is needed are methods, systems, and devices to overcome the above technical problems.

### BRIEF SUMMARY

The present disclosure provides technical solutions to overcome the above problems.

Systems, devices, and methods for automated approval of claim requests for solicited procedures are disclosed.

In an embodiment, a system includes an audit manager and an attention-based neural network. A computer-readable memory stores tuning parameters and a set of risk level thresholds. A database is configured to store training data including fixed length and variable length data. Fixed length data includes features and a target label. Variable length data includes medical procedure code approval history data. Validation data and operation data may also be stored in the database. The audit manager is configured to output an approval indication and rejection probability score for each solicited procedure according to a selected risk level threshold in the set of risk level thresholds. The attention-based neural network is trained according to features and a target label in the fixed length data and medical procedure code approval history data in the variable length data.

In further features, the attention-based neural network is configured to output the tuning parameters corresponding to the trained attention-based neural network. The audit manager is configured to apply validation data to the trained attention-based neural network to determine the set of the risk level thresholds.

In one embodiment, the audit manager is configured to, during an operation on a set of claim requests, select a risk level threshold for a set of claim requests and access solicited procedure data (X) for each claim request. The audit manager is further configured to determine historical procedure data (H) associated with the accessed solicited procedure data and feed the solicited procedure data (X) and determined historical procedure data (H) into the trained attention-based neural network to obtain a rejection probability score. The audit manager is further configured to compare the obtained rejection probability score to the selected risk level threshold and output an approval indication for each claim request based on the comparison.

In further features, the audit manager is configured to output the obtained rejection probability score for each claim request. In training, the audit manager is configured to feed training data to the attention-based neural network, the training data including fixed length data including features and a target label and variable length data including medical procedure code approval history data, and receive a rejection probability score from the attention-based neural network. The audit manager is further configured to determine an approval indication based on rejection probability score. In a further embodiment, the audit manager is configured to compare determined approval indication with target label in training data; adjust tuning parameters of attention based neural network based on rejection probability scores and determined approval indication until training condition met; and store set of tuning parameters in memory when training is complete.

In a further embodiment, the attention-based neural network is configured to, during training, determine a fixed length context vector C. The fixed length context vector C is based on the fixed length data and variable length data in the training data fed by the audit manager to the attention-based neural network. Also, in further features, attention-based neural network is configured to generate a fixed length attention data sequence A based on a concatenation of context vector C and associated solicited procedure data. The attention-based neural network is further configured to feed generated fixed length attention data sequence A into a dense layer of a neural network to obtain a rejection probability score for output to the audit manager.

In still further embodiments, computer-implemented methods for automated approval of claim requests for solicited procedures including fixed length and variable length data are provided. A non-transitory computer-readable medium for automating approval of claim requests for solicited procedures including fixed length and variable length data is also described.

Further embodiments, features, and advantages of this invention, as well as the structure and operation and various embodiments of the invention, are described in detail below with reference to accompanying drawings.

### BRIEF DESCRIPTION OF THE DRAWINGS/FIGURES

The accompanying drawings, which are incorporated herein and form part of the specification, illustrate the present disclosure and, together with the description, further serve to explain the principles of disclosure and to enable a person skilled in the relevant art to make and use the disclosure.

FIG. 1 is a system for providing automated claim approval decisions according to an embodiment of the present invention.

FIG. 2 is a flowchart of a process for initializing an automated claim approval decisions system according to an embodiment of the present invention.

FIG. 3 is a flowchart of a process for operating an automated claim approval decisions system according to an embodiment of the present invention.

FIG. 4 is a flowchart of a process for training an attention-based neural network for approval indication according to an embodiment of the present invention.

FIG. 5 is a flowchart of a process for determining context vectors with an attention-based neural network according to an embodiment of the present invention.

FIG. 6 is a diagram of an attention-based neural network according to an embodiment of the present invention.

FIG. 7A is a diagram illustrating solicited procedure data and historical procedure data according to an example of the present invention.

FIG. 7B is a diagram illustrating generating a context vector based on the solicited procedure data and historical procedure data of FIG. 7A according to an example of the present invention.

FIG. 7C is a diagram illustrating generating a fixed length attention data sequence based on a concatenation of the solicited procedure data and context vector according to an example of the present invention.

FIG. 7D is a diagram illustrating computing column weights with attention along with row weights for column/feature selection according to an embodiment of the present invention.

FIG. 8A is a line graph illustrating an optimum cut-off point determined in a scenario 1 test run of a model according to an embodiment of the present invention.

FIG. 8B is a line graph illustrating an optimum cut-off point determined in a scenario 2 test run of a model according to an embodiment of the present invention.

FIG. 8C is a line graph illustrating an optimum cut-off point determined in a scenario 3 test run of a model according to an embodiment of the present invention.

FIG. 9 shows a probability of rejection distribution for procedures approved by a system/auditor in a test run.

FIG. 10 shows a probability of rejection distribution for procedures rejected by a system/auditor in a test run.

FIG. 11 shows a confusion matrix with a 1% rejection cutoff point in a test run.

FIG. 12 shows a probability of rejection distribution for procedures rejected by a model in a test run where were not audited.

FIG. 13 shows a bar graph of model approved and rejected counts for procedures which were audited and approved by the model in a test run.

FIG. 14 is a diagram showing an example of eleven tables in a relational database upon which nested attention is applied in a further embodiment of the present invention.

FIGS. 15-20 are diagrams illustrating example displays in a user-interface to control review of automated pre-audit approval decisions in an embodiment of the present invention. FIG. 15 shows a display of data relating to an example solicited procedure being reviewed in a pre-audit along with approval controls. FIG. 16 shows a display of data including risk level relating to an example solicited procedure being reviewed in a pre-audit along with approval controls. FIG. 17 shows a display illustrating categories of pending solicited procedures grouped by level of risk determined in a pre-audit along with approval controls. FIG. 18 shows a display for navigating data relating to guias (claim requests). FIG. 19 shows a display illustrating categories of pending solicited procedures grouped by level of risk determined in a pre-audit along with approval controls. FIG. 20 is a display of a results dashboard in an embodiment of the present invention.

The drawing in which an element first appears is typically indicated by the leftmost digit or digits in the corresponding reference number. In the drawings, like reference numbers may indicate identical or functionally similar elements.

### DETAILED DESCRIPTION OF EMBODIMENTS

The present disclosure describes systems, devices, and methods for computer-implemented machine learning in request approval decisions. In embodiments, a system includes an audit manager coupled to an attention-based neural network. In one embodiment, an attention-based neural network includes a scalar dot product attention neural network. In a further embodiment, an attention-based neural network includes a scalar dot product attention neural network coupled to a concatenator unit. A dense layer is coupled between the concatenator unit and a sigmoid function unit.

A number of features and advantages are described. The inventors recognized and applied an attention-based neural network for the first time to computer-implemented machine learning in request approval decisions or auditing thereof. In one feature, an attention-based neural network provides an attention mechanism that can be used to efficiently learn useful, problem dependent features from relational databases. An attention-based neural network can intelligently resolve one-to-many relationships by learning to focus on rows from related tables which the neural network considers important for predicting the data labels. By using an attention-based neural network and creating fixed length context vectors, systems and methods herein can convert fixed length and variable length data to fixed length data for machine learning. Also an attention-based neural network herein may be far more amenable to parallelization than other neural network techniques, such as, inherently sequential recurrent layers. This can also lead to shorter training times.

### Terminology

The term “request” refers to a request for approval of a solicited procedure. A request may include, but is not limited to, a request for pre-authorization provided by a patient or a doctor on behalf of a patient to an insurer. For example, a request for pre-authorization may be a guia as used in providing health care in Brazil. A request may also include an insurance claim or claim.

The term “solicited procedure” refers to a procedure undergoing review for approval. A solicited procedure may include, but is not limited to, a medical procedure, task, supply item, or other expense requiring approval by an insurance carrier, medical provider, government, business, or other entity.

The term “attention-based neural network” refers to one or more computer-implemented neural networks having an attention-based mechanism. An attention-based neural network may include but is not limited to a scalar dot attention neural network or a hierarchical attention network.

The term “model” as used herein refers to a computer-implemented model, and is used interchangeably with the term “attention-based neural network” as described herein.

Embodiments refer to illustrations described herein with reference to particular applications. It should be understood that the invention is not limited to the embodiments. Those skilled in the art with access to the teachings provided herein will recognize additional modifications, applications, and embodiments within the scope thereof and additional fields in which the embodiments would be of significant utility.

In the detailed description of embodiments that follows, references to “one embodiment”, “an embodiment”, “an example embodiment”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to effect such feature, structure, or characteristic in connection with other embodiments whether or not explicitly described.

### Automated Claim Approval System

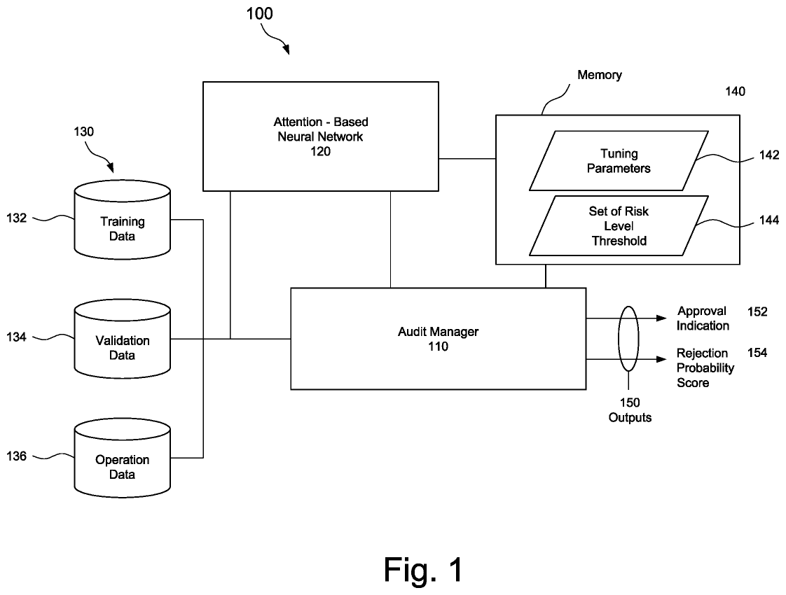

FIG. 1 shows a computer-implemented system 100 for providing automated claim approval decisions according to an embodiment of the present invention. System 100 includes an audit manager 110, an attention-based neural network 120, one or more databases 130, and memory 140. Audit manager 110 is coupled to attention-based neural network 120, database 130 and memory 140. Attention-based neural network 120 is also coupled to database 130 and memory 140.

Database 130 includes training database 132, validation database 134, and operation database 136 to store training data, validation data and operation data, respectively. Training data 132 includes fixed length data for features and a target label and variable length data for medical procedure code approval history data. Validation data 134 includes data for validating training of attention-based neural network 120 with respect to the target label. Validation data 134 may include accepted historical data and audit decisions for the target label made by human experts or other validated sources. Operation data 136 includes solicited procedures data and historical procedures data. Memory 140 stores tuning parameters 142 and a set of risk level thresholds 144.

In embodiments, system 100 including audit manager 110, attention-based neural network 120, and memory 140 as described herein can be implemented on or more computing devices. Audit manager 110 and attention-based neural network 120 can be implemented in software, firmware, hardware or any combination thereof on one or more computing devices. Memory 140 may be any type of computer-readable memory. Database 130 may be any type of relational database implemented on one or more data storage devices at the same or different locations. A database storage manager may be control access to one or more database 130 including database 132, 134, and 136.

Example computing devices include, but are not limited to, any type of processing device including, but not limited to, a computer, workstation, distributed computing system, embedded system, stand-alone electronic device, networked device, mobile device (such as a smartphone, tablet computer, or laptop computer), set-top box, television, console, kiosk, or other type of processor or computer system having at least one processor and computer readable memory. In further embodiments, system 100 as described herein can be implemented on a server, cluster of servers, server farm, or other computer-implemented processing arrangement operating on or more computing devices. Computing devices may be communicatively coupled across a network, such as, a local area, medium area or wide area network (e.g., the Internet).

In one embodiment, system 100 may be coupled to or integrated with a data platform such as the CAROL platform available from TOTVS Labs, Inc. System 100 may also include application programming interfaces (APIs) for coupling to remote services. A platform configured to support system 100 may also be implemented as a software-as-a-service (SaaS), platform as a service (PaS), or other web enabled service. In one embodiment, system 100 (including audit manager 110) may be accessed through a browser or through native application supporting web protocols to enable a user to provide further input and control or receive outputs from system 100 for display or storage. Audit manager 110 is operable to provide control for system 100 and components 120, 130 and 140. Audit manager 110 may communicate with one or more remote computing devices over a network and send one or more outputs 150. In embodiments, audit manager 110 may communicate with an application on a remote computing device. The application may be an application installed on the remote device or a web application accessed through a browser installed on the remote device. A user-interface may be provided on the remote device to allow a user to provide inputs and receive outputs through one or more I/O devices, such as, a display device, touch screen, keyboard, mouse, touchpad, microphone, speaker, tactile device, or other type of I/O device.

In operation, audit manager 110 is configured to output an approval indication 152 and rejection probability score 154 for each solicited procedure according to a selected risk level threshold selected from the set of risk level thresholds 144.

During training, attention-based neural network 120 is an attention-based neural network trained according to the training data 132. For example, attention-based neural network 120 is trained according to the training data including the features and target label in the fixed length data and medical procedure code approval history data in the variable length data. Attention-based neural network 120 is configured to output tuning parameters 142 corresponding to the trained attention-based neural network for storage in memory 140. Audit manager 110 is configured to apply validation data 134 to the trained attention-based neural network 120 to determine the set of the risk level thresholds 144.

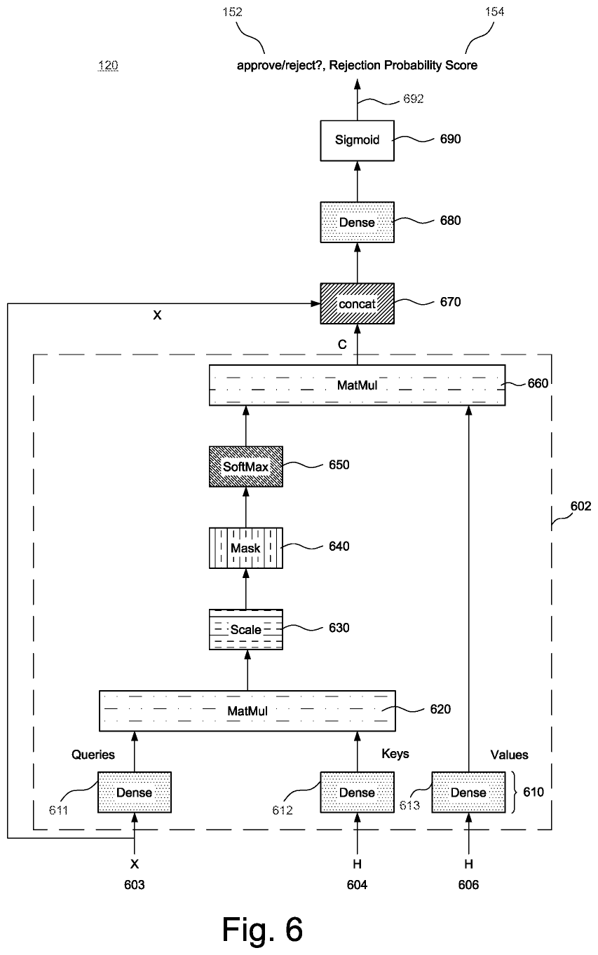

In an embodiment, attention-based neural network 120 may include a scalar dot product attention neural network as described in US Pat. Publ. Appl. No. 2019/0392319A1 to Shazeer et al. incorporated in its entirety herein by reference. FIG. 6 shows an attention-based neural network 120 in further detail according to an embodiment. As shown in FIG. 6, attention-based neural network 120 includes a scalar dot product attention neural network 602, concatenation unit 670, dense layer 680, and sigmoid function unit 690. Scalar dot product attention neural network 602 is coupled to concatenation unit 670. Concatenation unit 670 is also coupled to dense layer 680, which is coupled to sigmoid function unit 690.

Scalar dot product attention neural network 602 has three inputs that receive solicited procedure data (X) 603, historical procedure data (H) 604, and historical procedure data (H) 606, respectively. Scalar dot product attention neural network 602 includes three dense layers 610, first matrix multiplication unit 620, scalar unit 630, mask 640, soft maximum unit 650, and second matrix multiplication unit 660. Scalar dot product attention neural network 602 outputs a context vector C 662 to concatenation unit 670. Concatenation unit 670 concatenates a sequence of bits of solicited procedure data (x) 603 and a corresponding context vector C 662 to obtain an attention sequence of bits A 672. Concatenation unit 670 outputs attention sequence of bits A 672 to dense layer 680. Dense layer 680 processes the attention sequence of bits A 672 to obtain an output 682. Output 682 is provided to sigmoid function unit 690. Sigmoid function unit 690 applies a sigmoid function to output 682 and generates an output 692. Output 692 is then applied to audit manager 110 for further processing to generate an approval/rejection indication 152 and a rejection probability score 154.

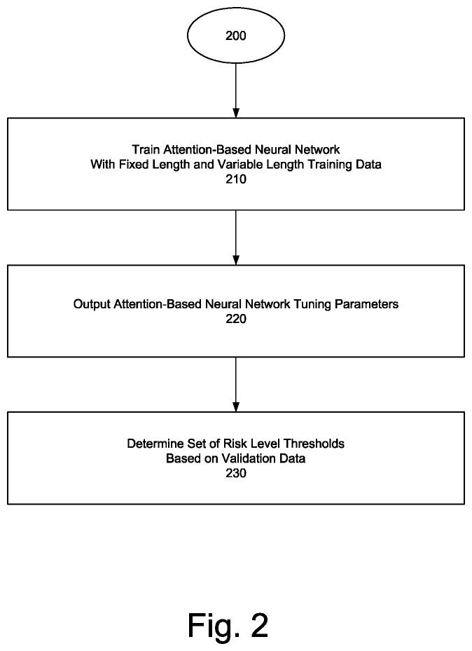

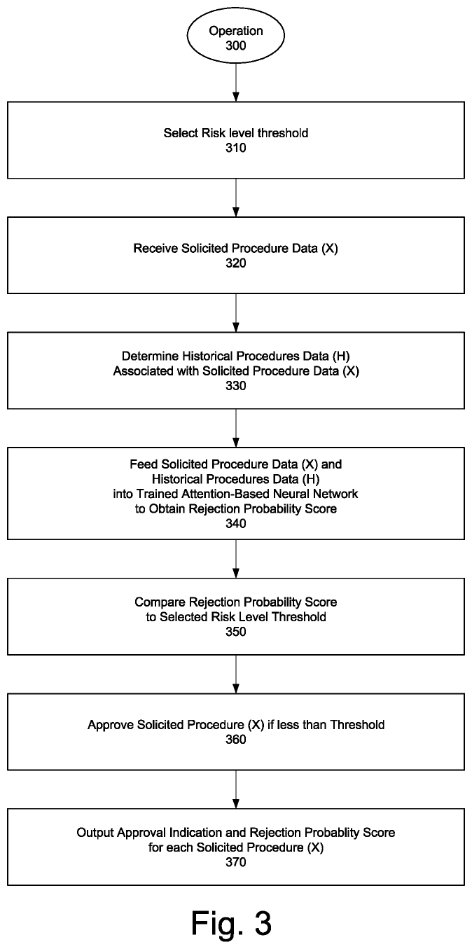

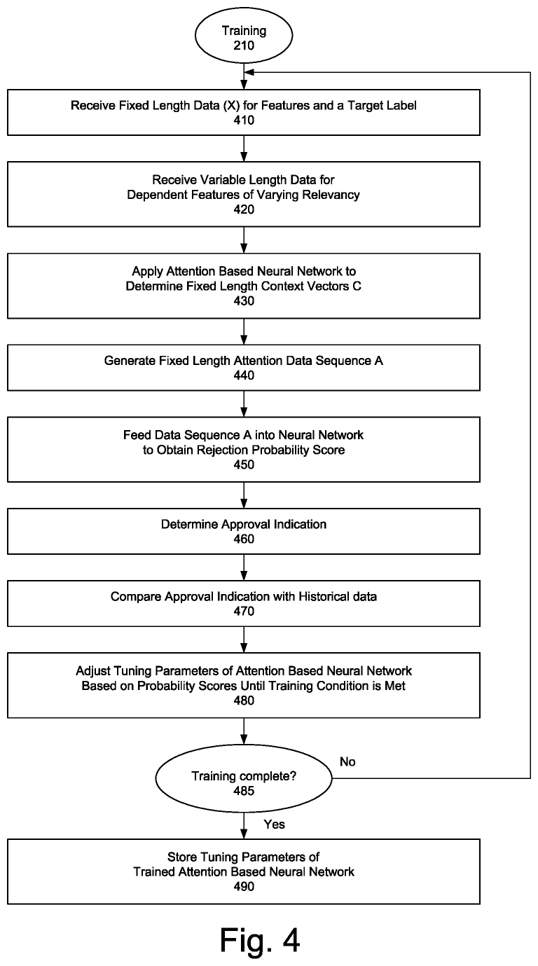

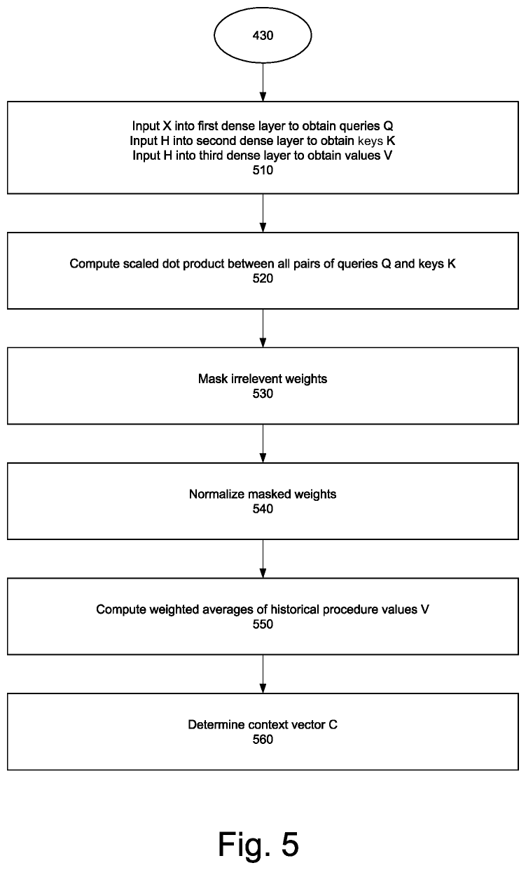

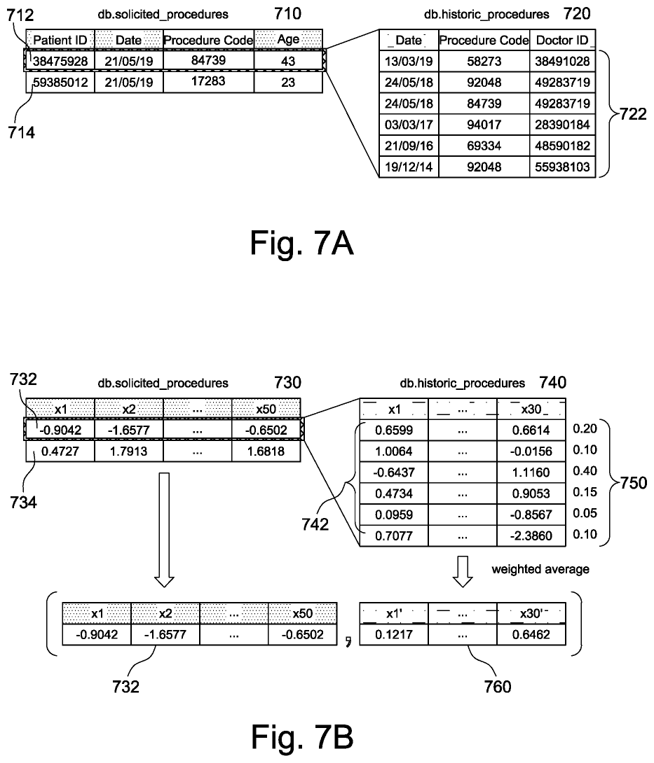

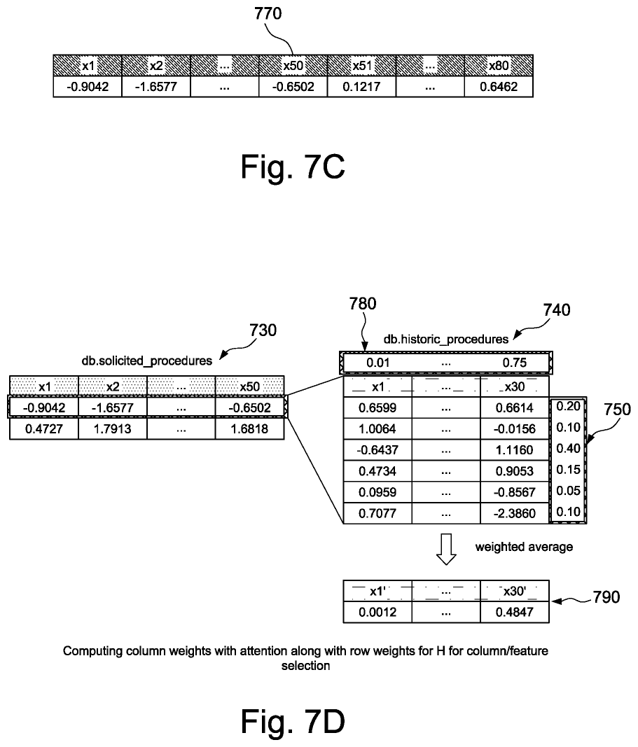

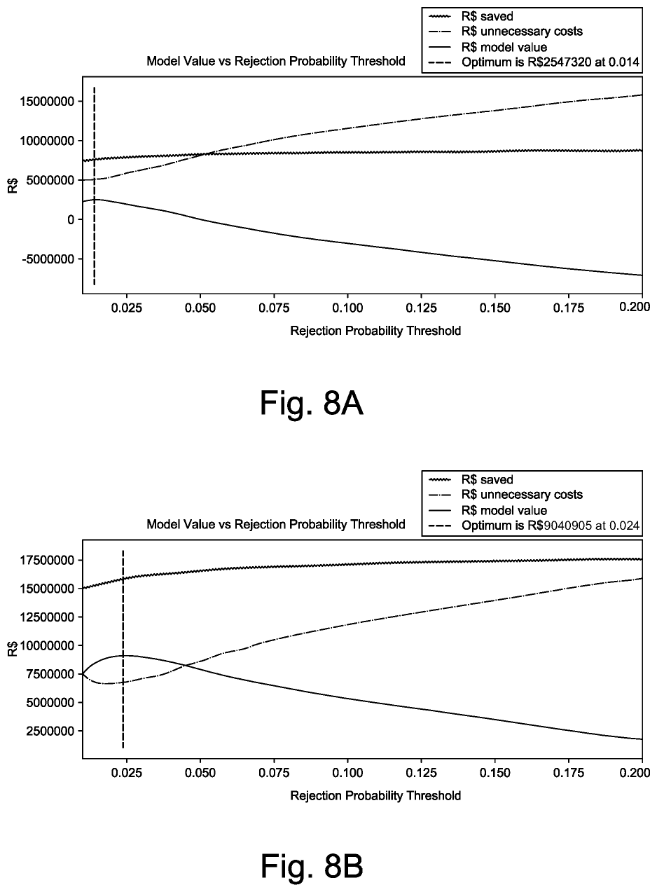

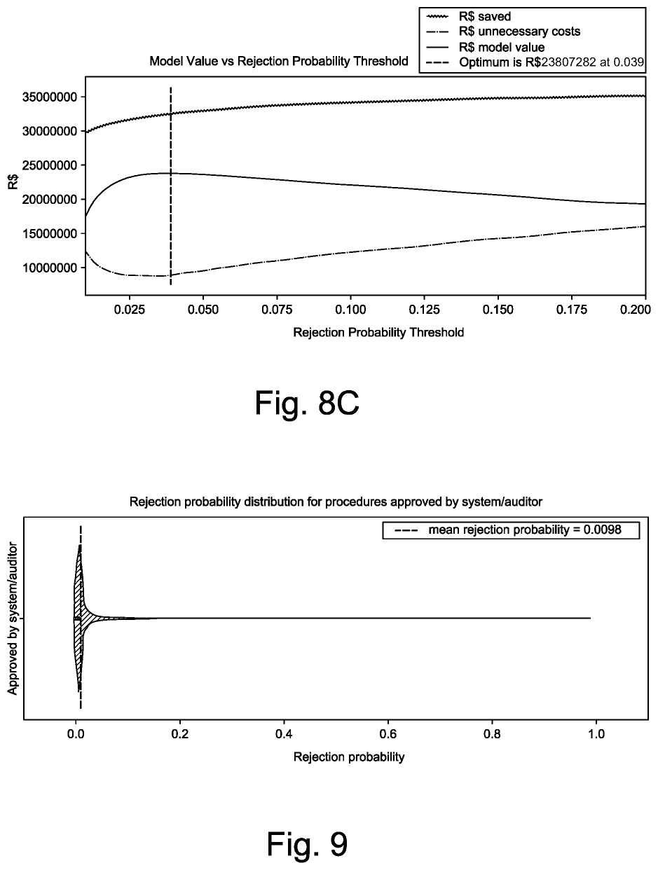

The operation of system 100 is described in further detail with respect to FIGS. 2-20. An initialization process for system 100 carried out under the control of audit manager 110 is described with respect to FIG. 2. The operation of audit manager 110 for determining claim approval decisions with a trained attention-based neural network 120 is described with respect to the process shown in FIG. 3. Training attention-based neural network 120 is described with respect to the process shown in FIGS. 4-5, the example attention-based neural network in FIG. 6, and example solicited procedure data and historical procedure data, row and column weights, context vector, and attention data sequence shown illustratively in FIGS. 7A-7D. Example results of claim approval decisions made by system 100 in an example test run are described with respect to FIGS. 8A-8C and 9-13.

### Initialization

FIG. 2 is a flowchart of a process 200 for initializing an automated claim approval decisions system 100 according to an embodiment of the present invention (steps 210-230). In step 210, attention-based neural network 120 is trained with fixed length and variable length data. Audit manager 110 may send a signal to attention-based neural network 120 to initiate training. Training is described further below with respect to FIG. 4.

In step 220, attention-based neural network 120 after training outputs tuning parameters 142 for storage in memory 140.

In step 230, audit manager 110 determines a set of risk thresholds 144 based on validation data 134. Each threshold may be set to correspond to a different respective amount of risk tolerance for an approval decision. Audit manager 110 outputs the set of risk thresholds 144 for storage in memory 140.

After initialization, operation may begin.

### Operation

FIG. 3 is a flowchart of a process 300 for operating an automated claim approval decisions system 100 according to an embodiment of the present invention (steps 310-370). Process 300 automates approval of claim requests for solicited procedures including fixed length and variable length data. In one embodiment, audit manager 110 performs steps 310-370. For brevity, the operation is described with respect to the example data shown in FIGS. 7A-7D. However, this example data is illustrative and not necessarily intended to be limiting.

In step 310, a risk level threshold is selected for a set of claim requests. Audit manager 110 may select a risk threshold from the set of risk thresholds 144. For example, audit manager 110 may select a risk threshold (low, medium, high) based on a preference set by a user or a default setting. A user may set a preference through a graphical user-interface or other control input to audit manager 110. In this way, a user may set a preference (low, medium, high) depending upon a particular application or need and audit tolerance.

In step 320, audit manager 110 accesses solicited procedure data (X) for each claim request. For example, audit manager 110 may query database 136 to retrieve solicited procedure data (X) for each claim request being processed.

Audit manager 110 determines historical procedure data (H) associated with the accessed solicited procedure data (step 330). Audit manager 110 may query database 136 to determine historical procedure data (H) associated with the accessed solicited procedure data (X).

In step 340, audit manager 110 feeds the solicited procedure data (X) and determined historical procedure data (H) into a trained attention-based neural network 120 to obtain a rejection probability score 154. For example, the output 692 of the sigmoid function unit 690 may be a numeric value representing the rejection probability score 154.

In step 350, audit manager 110 compares rejection probability score 154 to the selected risk level threshold. In step 360, audit manager 110 approves a solicited procedure (X) based on the comparison. For example, a solicited procedure (X) may be approved when the rejection probability score 154 is less than the selected risk level threshold.

Finally, in step 370 audit manager 110 outputs an approval indication 152 (according to the result of approving step 360) and a rejection probability score 154 (obtained in step 340) for each claim request. In one embodiment, an approval indication 152 in step 370 completes an automated approval decision of a solicited procedure request.

In an alternative embodiment, steps 350-360 may be carried out by attention-based neural network 120. For example, a comparator may be coupled to the output of sigmoid function unit 692. The comparator may compare the obtained rejection probability score 154 to the selected risk level threshold. An approval indication 152 to approve or reject based on the comparison may then be output by attention-based neural network 120 to audit manager 110.

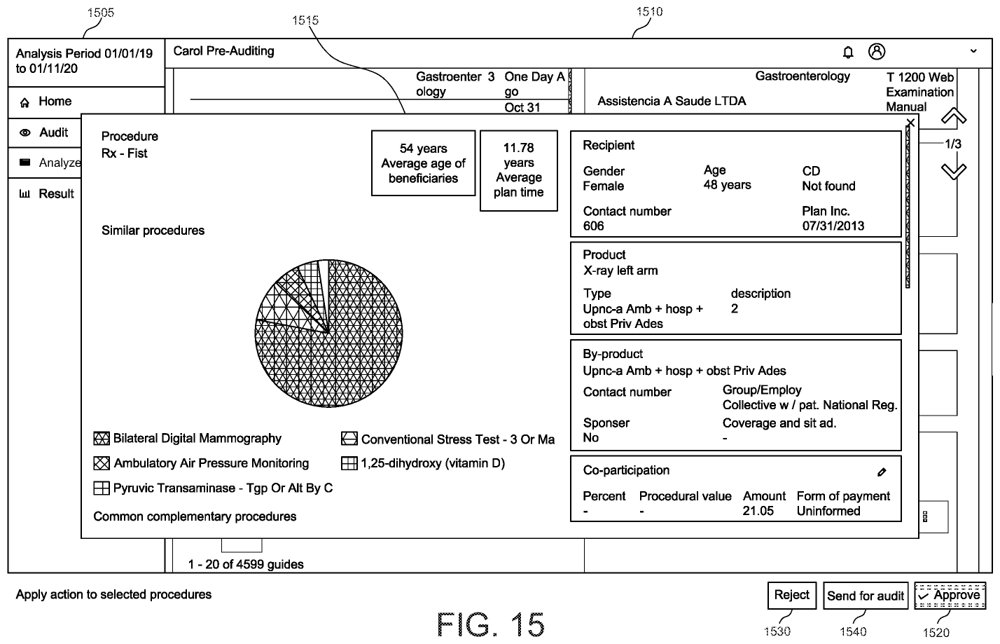

In another embodiment, steps 310-370 are carried out as part of a pre-audit. An approval indication in step 370 is part of the pre-audit of a solicited procedure request. With a pre-audit, audit manager 110 allows further control before an automated approval decision of a solicited procedure request is accepted. In this way, audit manager 110 enables a user or administrator to indicate approval for solicited procedures approved by trained attention-based neural network 120. In one feature, audit manager 110 may provide one or more displays and user-interface controls to enable a user to select whether to approve the solicited procedures approved in the pre-audit process. Examples of displays and user-interface controls that may be provided by audit manager 110 to control and management of pre-audit operations are described in further in detail below with respect to FIGS. 15-20.

### Training

FIG. 4 is a flowchart of a process 400 for training an attention-based neural network 120 for approval indication according to an embodiment of the present invention (steps 410-480). In an embodiment, process 400 may be initiated in step 210 in response to a signal from audit manager 110 to attention-based neural network 120. Attention-based neural network 120 is trained according to training data in database 132. The training data includes features and target label in the fixed length data and medical procedure code approval history data in the variable length data.

In one embodiment, steps 410-490 are performed by attention-based neural network 120. Audit manager 110 may send a control signal to attention-based neural network 120 initiate training step 210 and may receive a signal from attention-based neural network 120 indicating when training is completed after step 490.

In step 410, attention-based neural network 120 receives fixed length data (X) for features and a target label. The features may be solicited procedure information such as, patient ID, data, procedure code, and age. A target label may be an indication of claim approval (yes/no). Such fixed length data for features and target label for a particular solicited procedure can be drawn from a row having fixed length columns. For these features and label, often only one row having fixed length columns is needed for a particular patient pertaining to the particular solicited procedure.

In step 420, attention-based neural network 120 receives variable length data including medical procedure code approval history data. In this way, variable length data may cover dependent features of varying length relevancy, such as, medical procedure code approval history of patients, where multiple rows of medical procedure code data are often needed for a particular patient pertaining to the particular solicited procedure. Attention-based neural network 120 may query or access the training data in steps 410-420 from database 132.

In step 430, attention-based neural network 120 is applied to the fixed length and variable length data received in steps 410-420 to determine a fixed length context vector C. FIG. 5 is a flowchart of a process for determining context vectors with an attention-based neural network 120 according to step 430 an embodiment of the present invention (steps 510-560). For brevity, the process is further described with respect to attention-based neural network 120 having a scalar dot attention neural network 602 as shown in FIG. 6.

In step 510, control proceeds to input X 603 into a first dense layer 611 of set of layers 610 to obtain queries Q, input H 604 into second dense layer 612 of set of layers 610 to obtain keys K, and input H 606 into third dense layer 613 of set of layers 610 to obtain values V. Initially all neural net tuning parameters may be set randomly. For example, if input X=solicited procedures data; H=historical procedures data, then a set of tuning parameters (Q, K, V, and f, g, h,) 142 may be as follows:

Q=f(X)

K=g(H) and

V=h(H);

where f, g, and h are feed forward dense neural nets (or alternatively, weight matrices that are learned). As shown graphically in FIG. 6, f, g, and h may be a respective dense layer 611, 612 or 613 in a set of three dense layers 610. For example, each dense layer 611-613 in the set of layers 610 may be a single neural linear layer of a neural network.

In step 520, a scaled dot product (Q ⋅ K) is computed between all pairs of queries Q and keys K (620, 630). For example, matrix multiplication unit 620 may multiply (e.g., calculate a dot product) of queries Q and a K_transpose of keys K output from respective dense layers 611, 612. Scalar unit 630 may then multiply the dot product by a scalar to obtain a scaled dot product.

In step 530, mask 640 applies a mask (also called a filter) to the scaled dot product to mask irrelevant weights and obtain masked weights for rows and/or columns.

In step 540, masked weights are then normalized (e.g., a softmax function unit 650 may apply a softmax function to the masked weights output from mask 640). For example, a softmax function may normalize masked weights to a range between 0 and 1 to facilitate use in a probability score.

In step 550, control proceeds to compute weighted averages of historical procedure values V. In one embodiment, a second matrix multiplication unit 660 may multiply masked weights output from softmax function unit 650 with historical procedure values V output from dense layer 613.

In step 560, a context vector C is determined based on the weighted averages computed in step 550. In this example, a context vector C is a set of bits (also called a sequence of bits). For example, context vector C may equal softmax(QK_T/scale)*V (mask omitted for clarity. K_T=K transpose).

This context vector C has a fixed length of bits and is further used to train attention-based neural network 120.

In step 440, control proceeds to generate a fixed length data sequence A. For example concatenation unit 670 may concatenate solicited procedure data X 603 with an associated received context vector C determined in step 430 and output from scalar dot product attention network 602. Solicited procedure data X 603 is fixed length and context vector C is fixed length. Concatenation forms a fixed length attention data sequence A. For example, A=concat(X+C) (number of context vectors=number of entries in X).

In step 450, control proceeds to feed data sequence A into a neural network (such as a dense layer 680 of attention-based neural network 120) whose output is coupled to sigmoid function unit 690 to obtain a rejection probability score 154. In one example, rejection probability score 154 is equal or substantially equal to a numerical value output of a sigmoid function applied in sigmoid function unit 690.

In one embodiment, dense layer 680 is a last layer of a trained neural network. This can be a dense layer in attention based neural network 120. Embeddings are used to compute similarities between procedures to be used by the auditor to audit that procedure.

In step 460, control proceeds to determine an approval indication 152 based on the obtained rejection probability score 154. For example, if the rejection probability score 154 is less than a threshold than an approval indication 152 may be set to approve; otherwise, it is set to reject or disapprove.

In step 470, control proceeds to compare the determined approval indication 152 in step 460 with a target label in training data (can be validation data or historical data). The target label in training data is a label (approval indication) that is validated or set by human experts or other validated sources. A match in this comparison indicates successful training condition is met.

In step 480, control proceeds to adjust tuning parameters of attention based neural network 120 based on the rejection probability scores 154 and determined approval indication 152 until a training condition met.

In step 485, a check is made of whether training is completed. For example, control may evaluate whether a predetermined number of epochs of solicited procedures (claim requests) in the training data are completed. In another example, an early stopping technique may be used to evaluate whether training is completed. For example, an early stopping technique using regularization to prevent overfitting in training may be used. See, e.g., Ian Goodfellow et al., Deep Learning, MIT Press Cambridge Mass. (2016), Section 7.8, pp. 239-245. If not, control proceeds to step 410 to process the next solicited procedure being evaluated for claim approval training. Otherwise, controls proceeds to step 490.

In step 490, control proceeds to store a set of tuning parameters 142 in memory 140 when training is complete. These stored tuning parameters are the values adjusted during training until the training condition is met (or all records are processed). For example, set of tuning parameters 142 may be the tuning parameters identifying (Q, K, V, and f, g, h,) obtained for the trained attention neural network 120. A set of risk thresholds 144 may also be determined and stored in memory 140.

### Set of Risk Thresholds

In an embodiment, system 100 may assign three risk levels; low, medium, and high. All these thresholds can be computed using validation data in database 134. For example, a separate validation set not used for any other tuning parameter or hyperparameter optimization may be used. The definitions of the low, high, and medium thresholds may be chosen as follows:

The high threshold is chosen such that rejecting all procedures with rejection probability greater than the high threshold would result in a recall of 0.95.

The medium threshold is chosen such that rejecting all procedures with rejection probability greater than the median threshold would result in a recall of 0.9.

The low threshold is the rest.

The solicited procedures flagged as medium and high risk will be used to compute system 100 performance using:
a. the number of procedures that were approved and were flagged with medium or high probability.
b. the number of procedures that were rejected and were flagged with medium or high probability.
c. the number of procedures that were rejected and were flagged with low probability.
d. the number of procedures that were approved and were flagged with low probability.

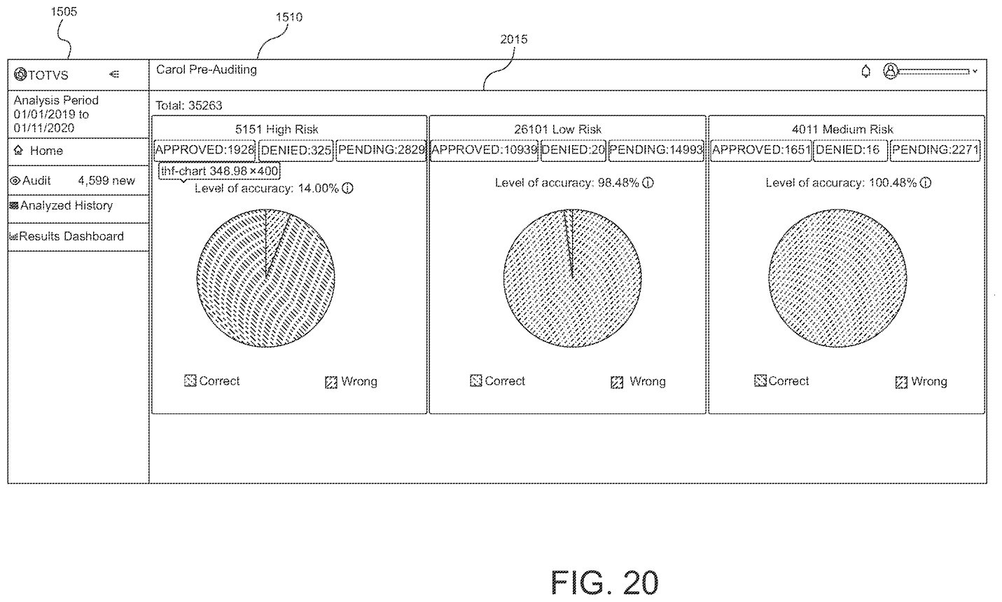

For example, FIG. 20 described further below shows examples of displays showing output results for solicited procedures evaluated with high, low, and medium thresholds.

This allows an insurer to approve in batch the solicited procedures with a low probability to be rejected. The low threshold of low probability is computed using the recall. System 100 performance may be computed by comparison with those procedures that were audited manually.

### EXAMPLES

FIG. 7A is a diagram illustrating solicited procedure data 710 and historical procedure data 720 according to an example of the present invention. Solicited procedure data 710 includes rows of fixed length data for four features (Patient ID, Date, Procedure Code, Age). In one example, each row corresponds to particular solicited procedure being evaluated for approval. For example, row 712 may include Patient ID, Date, Procedure Code, and Age for a first patient. Row 714 may include Patient ID, Date, Procedure Code, and Age for another patient.

Historical procedure data 720 includes variable length data (that is, one or more rows of fixed length data) associated with solicited procedure data 710. Because historical data often has relevant data for multiple procedures corresponding to a particular patient it can be of varying length. As shown in the example of FIG. 7A, historical procedure data 720 may include variable length data 722 made up of six (6) rows of data for three features (Date, Procedure Code, Doctor ID) all of which are associated with the data in row 712 of a particular patient.

FIG. 7B is a diagram illustrating generating a context vector C based on the solicited procedure data and historical procedure data of FIG. 7A according to an example of the present invention. Table 730 shows rows of weighted averages for solicited procedures data processed by attention based neural network 120. Row 732 is a row of fixed length data with weighted averages for a solicited procedure for a first patient. The row has 50 columns representing weights corresponding to 50 parts relating to the four features (Patient ID, Date, Procedure Code, Age) of row 712 fed to attention based neural network 120 by audit manager 110. Row 734 shows similar data obtained for row 714 fed to attention based neural network 120 by audit manager 110.

Table 740 shows historical procedures data processed according to a set of weights 750. To illustrate how a context vector C (row 760) is determined, table 740 shows rows 742 of weighted historical procedures data (variable length) processed by attention based neural network 120. Rows 742 are rows of fixed length data corresponding to historical procedure data for a solicited procedure for a first patient weighted by weights 750. Each row in rows 742 has 30 columns representing 30 weighted parts relating to three features (Date, Procedure Code, Doctor ID) of row 722 fed to attention based neural network 120 by audit manager 110.

By taking a weighted average of rows 742, a context vector C (row 760) is obtained. FIG. 7B further illustrates how row 732 of fixed length data and row 760 of fixed length may be concatenated to form a fixed length attention data sequence (one row of 50 columns).

FIG. 7C is a diagram further illustrating a table 770 having a fixed length attention data sequence A based on a concatenation of the weighted solicited procedure data 732 and context vector 760 according to an example.

### Claim Approval Decisions

Consider claim approval decisions using system 100 and method 300 and a set of risk thresholds 144 in an example test run. The approval decisions were made for solicited procedures data presenting guias. A guia is a request for pre-authorization in Brazilian health care, from a doctor to an insurer, to perform one or more medical procedures and/or utilize medical supplies. For every new request, a new guia is created (even if the request is related to ongoing treatment). The request refers to a set of procedures and supplies being requested for approval by an insurance company.

Pre-authorization should be done before any treatment is carried out. A conventional approach was for the insurer to use a set of rules to decide whether to authorize the request or send it to the auditor for further analysis. If it goes to the auditor he/she will manually analyze every procedure and supply of the guia and decide whether or not to contest each one. Although they make a decision with regards to each individual procedure/supply, the context of the guia, as a whole, influences their decision. The decision on the guia is made only after analyzing all associated procedures and supplies. The guia can be either fully or partially authorized; if the auditor contests a procedure or supply in the guia, we said it was partially authorized. A guia can be related to other guias requested in the past. The auditor considers one or more factors when deciding whether to approve the guia or not, including the following:

if the patient is covered for all procedures in the request,

if the patient's plan has expired,

if all the medical supplies requested are indeed required to carry out the procedures.

The auditor takes into account previous related guias and even consider requests which don't appear to have a direct link to the current request. When the auditor contests a procedure or supply, a glosa is created. One or several glosas can be created for any procedure or supply. The past glosas should be considered by the auditor to decide whether to approve the request or not. All of this can be time-consuming and cost-prohibitive in practice.

Depending on the nature of a procedure/supply, a decision if either to approve or reject a procedure must be given up to 72 h after the request has been made. Due to the number of requests an insurer receives it is not possible to audit all of them. So, there are several rules that will automatically approve or reject the requests. In many cases, for cheap procedures, insurers will automatically approve, no questions asked thereby sacrificing accuracy and increasing fraud risk.

In embodiments of the present invention described herein, a number of technical advantages in claim approval decision making are realized with machine learning, attention based neural networks, scalability, automated feature engineering for fixed length and variable length data, and faster, accurate computer-implemented decision making for claim approvals. Other significant advantages such as reduced cost, less work, and increased savings from approval decisions are also achieved.

In one embodiment, automated claim approval decisions system 100 and method 200 may reduce the number of medical forms (guias) that are sent to the auditor. For example, system 100 may be set to have the authority to approve requests, but not to reject them. A Brazilian health regulatory agency may establish that the insurer must justify when a procedure is rejected. Any requests which are not approved by system 100 can be sent to the auditor for further analysis. Every request approved by automated claim approval decisions system 100 saves the insurer the amount it would have paid to the auditor. However, cost is incurred when system 100 approves a request that would have been rejected by the auditor.

Consider this example about insurance companies. The number of requests audited can vary a lot among different insurers, but may be typically between 8% to 20% of all the requests. A medium to a large-size insurer can have between 500,000 to 1,000,000 procedures/supply to audit per month. The cost to manually audit a single procedure depends on the type of procedure, but one can estimate for this example that it can be from R $4.00 to R $15.00 (units in Brazilian real). A medium to large size insurer can have from 400,000 to 800,000 lives (number of clients/patients).

To put more simply, assume an insurance company receives 100 claims per day. Assume it costs 1 US dollar for the insurance company to manually arrive at an approve/reject decision per claim. Because of a lack of manpower, the insurance company automatically approves 50 claims and only makes a manual decision on the remaining 50. So, it costs the company 50 US dollars a day to process claims. Of the remaining 50 claims, let us say, historically it has been found that 80% (40) are approvals and 20% (10) are rejects. So, in a day, there are 50+40=90 approvals and 10 rejects with a cost of 50 dollars for the insurance company. However, in embodiments automated claim approval decisions system 100 and method 300 can automatically (and correctly) approve a claim with high accuracy. These automated claim approval decisions are made for approvals and not for rejections. So returning to this example, assume automated claim approval decisions system 100 flags 75 claims as definite approvals per day. Then the insurance company has to process only the remaining 25 claims. The total cost per day reduces to 25 dollars, or a 50% savings.

As a result in one feature, automated claim approval decisions made in system 100 and method 300 with machine learning on guia (claim/request) approvals can reduce the total load and drastically decrease the time to process (esp. approvals). In one embodiment, system 100 and method 300 are configured to approve requests (those with a very low rejection probability), but not to reject any requests. This avoids “false rejects” which can be relatively harmful (both for the patient's health as well as the insurer's reputation) compared to “false approvals” which only incur a small cost for the insurer. In this way, insurers can use automated claim approval decisions made in system 100 and method 300 with the assurance that claims are not incorrectly rejected. Further a set of risk thresholds are provided to allow an insurer to further configure risk tolerance for particular types of approval decision making. Also in a further feature, automated claim approval decisions made in system 100 and method 300 may not reject a guia, but gives a rejection probability score 154 or recommendation. Auditors and/or insurers handling rejections may use a rejection probability score 154 or recommendation as one of the data points for arriving at a reject/approve decision.

In a further feature, embodiments described herein can help address system fraud. Insurance companies may pay special attention to claims with a high rejection probability score 154. If the rejection probability score 154 is high, insurers may elect to always manually audit the claim. For example in one test run, the inventors evaluated 889,176 procedures that the model would not approve in this scenario, 475,340 were not audited and ended up being approved. 6,142 of these procedures have a probability of rejection greater than 20%, with a total cost of R$584,367. Some of them could be possible frauds and the model would have caught them.

Training may provide further advantages. System 100 receives only three (3) inputs: <patient age, patient sex, medical_procedure_code_requested> to make an “approve” decision. System 100 (and in particular attention based neural network 120) may then train from a historical table with 4 columns <patient age, patient sex, medical_procedure_code_requested, human_decision> and 1 million rows. Each row is a specific value for some patients. The table (stored in database 132) may look like (Table 1):

TABLE ONE

| Patient | | | Human |
|---|---|---|---|
| Age | Sex | Medical_Procedure_Code_Requested | Decision |
| 22 | M | 45634 | Approve |
| 67 | F | 34234 | Approve |
| 15 | M | 55543 | Reject |
| 45 | F | 45345 | Approve |

<999,996 More Rows>

The first 3 columns are features. The last column (human decision) is the target. To help humans, the target is what an attention based neural network 120 has to learn to predict automatically after training given the first 3 columns (<patient age, patient sex, medical_procedure_code_requested>). After training from historical 1 million records, system 100 with a trained attention based neural network 120 can be used in place of human decisions.

Not only may attention based neural network 120 train from tables that have a fixed number of columns (4 in this case=3 features and 1 target), but in a further feature, attention based neural network 120 may also train from tables that have variable length data. For example, one or more tables having patient medical procedure code approval history (all the medical_procedure_codes requested by the patient in the past and the insurance company decision for those procedures) may be used which may be very relevant information.

Often patients have variable length medical procedure code approval history. For example, assume a first patient has no procedures requested in the past. A second patient has 10; a third patient has 3; a fourth patient has 26, and so on. A table like Table One expanded to include this medical procedure code approval history then becomes: 4+0 columns for the first patient, 4+10 columns for the second patient, 4+3 columns for the third patient, 4+26 columns for the fourth patient, and so on.

Conventional machine learning models only like to train from tables that have a fixed number of columns. System 100 including attention-based neural network 120 converts variable-length columns/features to fixed-length columns/features in training and solves this problem. This also increases accuracy and allows relevant information like medical procedure code approval history to be used in machine learning.

Also, all medical procedure code approval history of the patient may not be relevant to make an approve/reject decision on the current requested medical procedure. For example, if the current medical procedure requested is for heart bypass surgery, perhaps the patient's dental procedure code approval history is less relevant compared to the patient's heart-related medical procedure approval history. System 100 by using attention-based neural network 120 allows machine learning to pay attention to only historical procedure claim requests of the patient that are most relevant. Weights and weighted averages are used to obtain a context vector. The context vector is a fixed length and incorporates attention to more relevant data parts. Attention based neural network 120 may also have multiple, nested attention layers that can learn from more complex databases and different tables or records located in the same or different databases.

These features are illustrative and not intended to be limiting. Other features may be used. For example, new features for machine learning using the glosa text for a given procedure/supply may be used. Features for procedures/supplies like brand, description, quantity may be added.

For brevity, embodiments are described with respect to request approval decisions and solicited procedures involving medical procedures. However, this is illustrative and not intended to limit the present invention. Other types of data in different applications may be used as would be apparent to a person skilled in the art given this description.

### Example Test Run

The inventors performed a test run of system 100 using an audit manager 110 and attention-based neural network 120 in an embodiment. Three scenarios (Scenarios 1-3) having respective cost of audit values R=5, 10, and 20 (Brazilian reals) were evaluated.

### Analysis

In this test run, 11,342,453 medical procedures were used as solicited procedure data (X). Attention-based neural network 120 (in particular, a scalar dot attention neural network) was trained as described herein to learn which procedures should be approved and which should be rejected in request approval decisions. The trained scalar dot product attention neural network for purposes of this test run is also referred to as a model.

The model makes the classification in based on the following information: patient's age, patient's sex, plan time, CID, procedure code, medical specialty, requester code, provider code, criticism, other procedures on the same tab, and patient's medical history. Data for this information from 2016 and 2017 was used as training data.

The inventors tested the model in operation on the 5,416,196 procedures that were requested using actual requests made in 2018. In the example test run, procedures with a value greater than R$10,000 were excluded as conservative insurers may not employ a model to work on procedures with such a high cost.

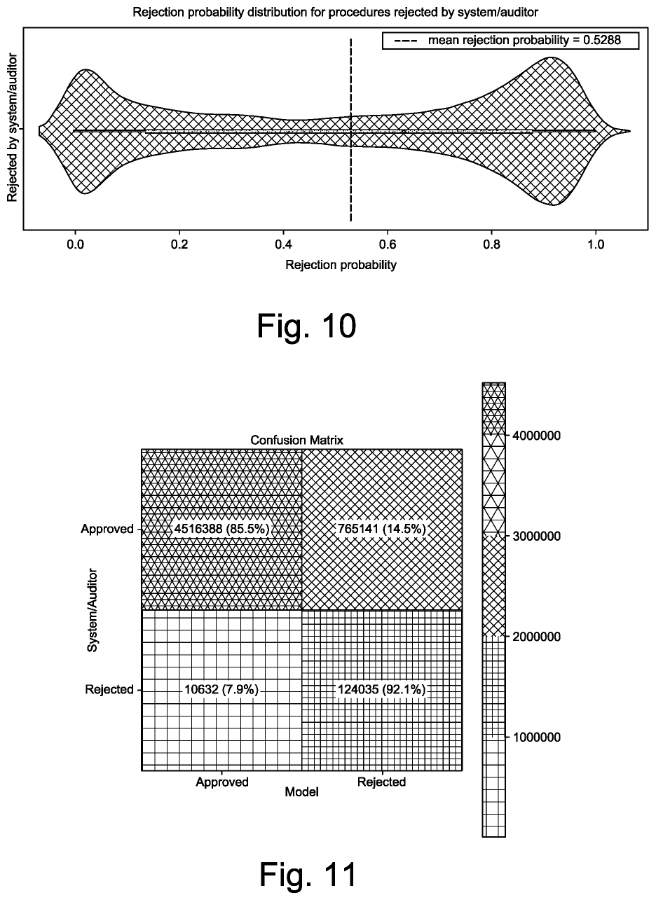

The model predicted a probability of rejection distribution as shown in FIG. 9 for procedures approved by a system/auditor. The model also predicted a probability of rejection distribution as shown in FIG. 10 for procedures rejected by a system/auditor. As evident in comparing the distributions, the model has learned to assign much higher rejection probabilities for procedures that have actually been rejected. The average probability of rejects was 52.88%, 54 times (54×) greater than the average probability of 0.98% of those approved.

As described above, one or more risk thresholds are set. This may be based on a rejection cut off point. For example, in practice, system 100 (or a user of system 100) may define a rejection cutoff point below which all procedures will be approved without going through the audit. Procedures that have a higher probability of rejection than the cutoff point will have to pass an audit.

FIG. 11 shows a confusion matrix with a 1% rejection cutoff point (or cut-off bridge). Thus, all procedures with a rejection probability of less than 1% would be approved by the model and the others would have to pass to audit. The confusion matrix shows where the model agrees and disagrees with results obtained in a real human audit reality.

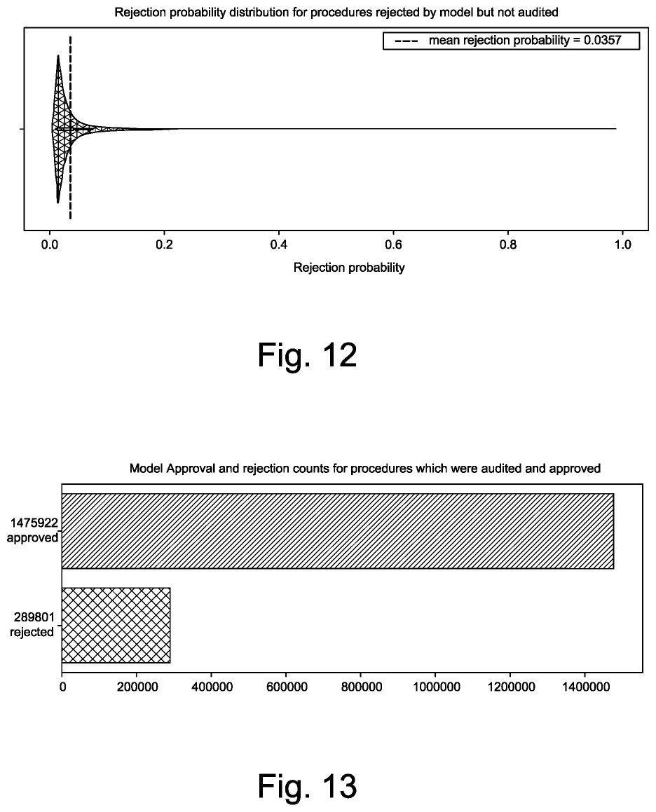

The model may be used to help detect fraud. For example, in the test run of the 889,176 procedures that the model would not approve (see right side of confusion matrix FIG. 11), 475,340 were not audited and ended up being approved. The distribution of the probabilities of rejection of these procedures is shown in FIG. 12. 6,142 of these procedures have a probability of rejection greater than 20%, with a total cost of R $584,367. In this case, some of them could be possible frauds and the model would have caught them.

1,823,007 (33.7%) of the 5,416,196 procedures requested in the 2018 data entered into a pending release/audit state at some point. Of those, 1,765,723 procedures (requests) were approved and 57,284 rejected. Of the 1,765,723 procedures that went into a pending release/audit state but ended up being approved, it is helpful to consider how many would have been approved by the model. FIG. 13 shows there are 1,475,922 procedures that the model could have approved without requiring an audit and the result would have been exactly the same.

To better understand cost savings that may be obtained by a model. For example, money may be saved when the model approved a procedure that previously would been audited less costs generated when a model approves a procedure that should not be approved or indicated a procedure to be audited that does not need an audit. Three scenarios 1-3 were considered. The frequency of each case is shown below when the rejection cut-off point is 1%.

In scenario 1, the average cost of an audit is assumed to be R$5. An optimum cutoff point is 1.4%. As shown in FIG. 8A, a plot of the model value over a range rejection probability threshold values has a maximum at or near 0.014 (1.14%). The model would have approved 4,696,701 (86.7%) of the 5,416,196 procedures, saving R $7,610,045 from audits. 12,589 (0.27%) of them would have been incorrectly approved, costing R$3,329,715. 346,602 procedures would have been indicated to be unnecessarily audited, costing R$1,733,010. The difference, the total value of the model for the 2018 data would have been R$2,547,319.

In scenario 2, the average cost of an audit is assumed to be R$10. The optimum cutoff point is 2.4%. As shown in FIG. 8B, a plot of the model value over a range rejection probability threshold values has a maximum at or near 0.024 (2.4%). The model would have approved 4,905,095 (90.6%) of the 5,416,196 procedures, saving R $15,792,950 from audits. 16,119 (0.33%) of them would have been incorrectly approved, costing R$4,781,295. 197,075 procedures would have been indicated to be unnecessarily audited, costing R$1,970,750. The difference, the total value of the model for the 2018 data would have been R$9,040,905.

In scenario 3, the average cost of an audit is assumed to be R$20. The optimum cutoff point is 3.9%. As shown in FIG. 8C, a plot of the model value over a range rejection probability threshold values has a maximum at or near 0.039 (3.9%). The model would have approved 5,037,609 (93.0%) of the 5,416,196 procedures, saving R $32,516,440 with audits. 19,740 (0.39%) of them would have been incorrectly approved, costing R $6,461,118. 112,402 procedures would have been indicated to be unnecessarily audited, costing R$2,248,040. The difference, the total value of the model for the 2018 data would have been R$23,807,282.

### Payoff Report

In a further embodiment, audit manager 110 may generate a payoff report. A payoff report may be generated for a potential user based on their historical data. For example, an insurer may upload their historical guias (e.g, from the past 1 year) and get an estimate of savings had they used automated claim approval decisions made in system 100 or method 200.

### Pre-Audit User-Interface

In embodiments, audit manager 110 may further provide a user-interface on a remote application to enable remote users or administrators using remote computing devices to review solicited procedures evaluated by a trained attention-based neural network 120 in a pre-audit. FIG. 15 shows a pre-audit control panel having a control menu 1505, data display panel 1510, and control buttons 1520, 1530, 1540. Control menu 1505 may include user interface elements enabling a user to select one or more displays relating to home, audit, analyzed history, and a results dashboard. Data display panel 1510 includes an area of a display for displaying pertinent data either within panel 510 or in one or more pop-up windows, tabs, or separate display panels, such as, separate panel 1515.

Panel 1515 displays data relating to an example solicited procedure being reviewed in a pre-audit for a particular recipient. Panel 1515 includes information identifying the procedure, the recipient (including name, gender, age, CID, contact number, health insurance plan). A chart of similar procedures is included. Summary information on product(s) and byproduct(s) is included. Further health insurance plan information, such as, co-participation information may be included. Control button 1520 enables a user to approve the solicited procedure. Control button 1540 enables a user to reject the solicited procedure. Control button 1530 enables a user to flag the solicited procedure for further audit. Control buttons 1520, 1530, 1540 are illustrative and other user-interface control elements (e.g., menus, sliders, dials) may be used to provide a control input through touch, voice, or other keyboard input.

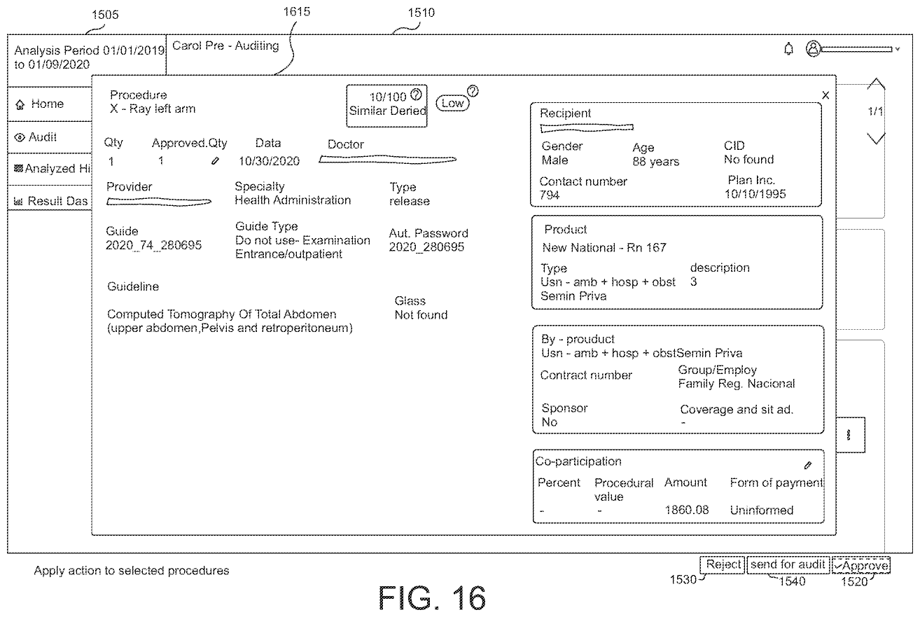

FIG. 16 shows a display panel 1615 including data on risk level relating to an example solicited procedure being reviewed in a pre-audit along with approval controls. The risk level, for example, may be high, medium or low rejection probability, depending upon the set of risk thresholds used by trained attention-based neural network 120 in the pre-audit. As shown in FIG. 16, if the solicited procedure was indicated as approved in step 370 and a rejection probability score was low, then a graphical indication such as a green “Low” image may be displayed. Pertinent pre-audit data on the solicited procedure may be displayed as well, such as, an indication of a ratio of similar denied procedures, quantity, approved quantity, date, doctor, speciality, health code (guia or guide), health code type (guide type), and guideline information. Other pertinent data to the solicited procedure may also be displayed (such as, recipient, product by-product, and co-participation information). In this way, a user in this pre-audit can easily review and verify the solicited procedure approved by trained attention-based neural network 120, and if the user agrees can select button 1520 to approve the solicited procedure.

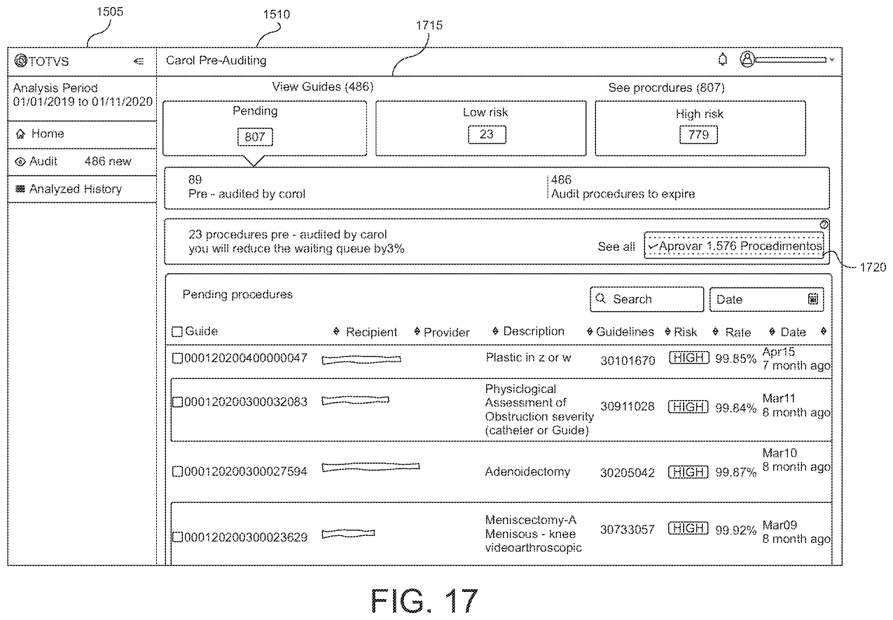

FIG. 17 shows a display panel 1715 illustrating categories of pending solicited procedures grouped by level of risk determined in a pre-audit. In this example, graphical indications are displayed indicating the number of pending solicited procedures processed to date in the pre-audit along with the number of those having a low risk and high risk rejection probability. In one feature, the group of procedures indicated as having a low risk, may be selected for approval. In this way, a user in this pre-audit can easily review and verify a group of low risk solicited procedures approved by trained attention-based neural network 120, and if the user agrees can select button 1720 to approve the solicited procedures. Display 1715 may also include other pertinent data to provide more context, such as, pre-audit workflow (e.g., number of solicited procedures already pre-audited, number of solicited procedures about to expire due to delay or untimeliness, and navigable listings on pending solicited procedures). Search controls (such as a search text window, search by date window, category sort controls, and selection boxes may be provided).

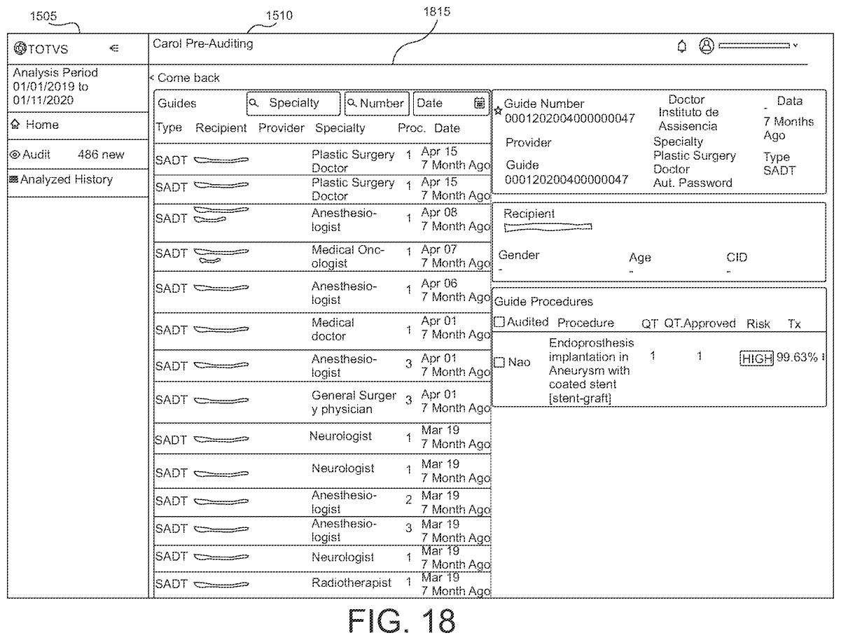

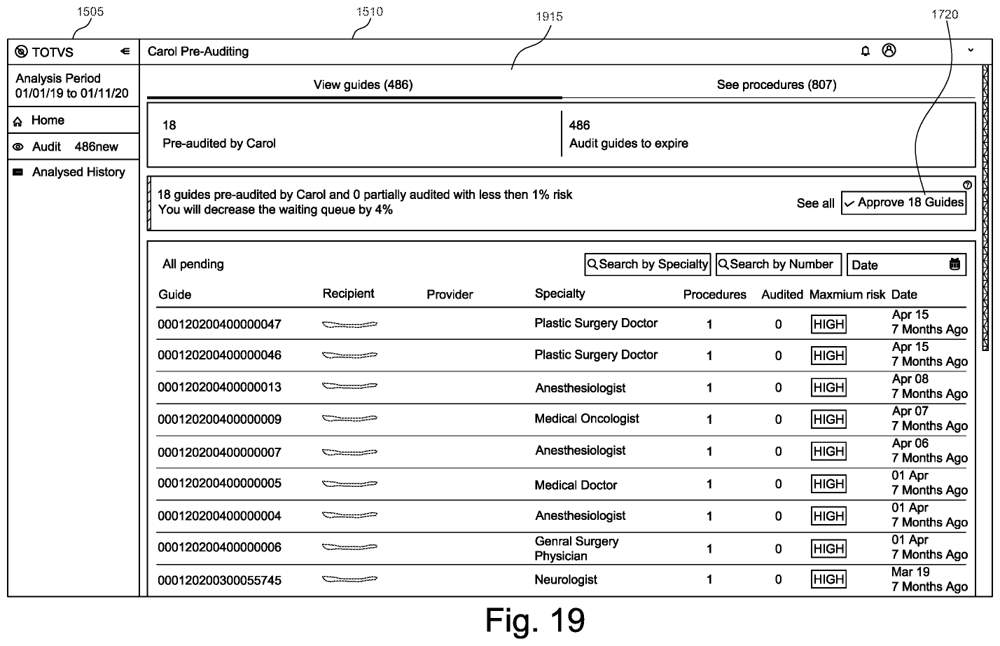

FIG. 18 shows a display panel 1815 for navigating data relating to guias (claim requests) labeled here for convenience as guides. FIG. 19 further shows a display panel 1915 illustrating categories of pending solicited procedures grouped by level of risk determined in a pre-audit along with approval controls. FIG. 20 is a display panel 2015 showing a results dashboard in an embodiment of the present invention. The results dashboard includes summary displays of results with respect to requests processed in a pre-audit having high, medium, and low risks. These results may include counts of requests approved, denied, and pending, and a level of accuracy for the model and a pie chart of the percentage of approval decisions found correct and wrong by an attention-based neural network 120 compared to human experts.

### Nested Attention

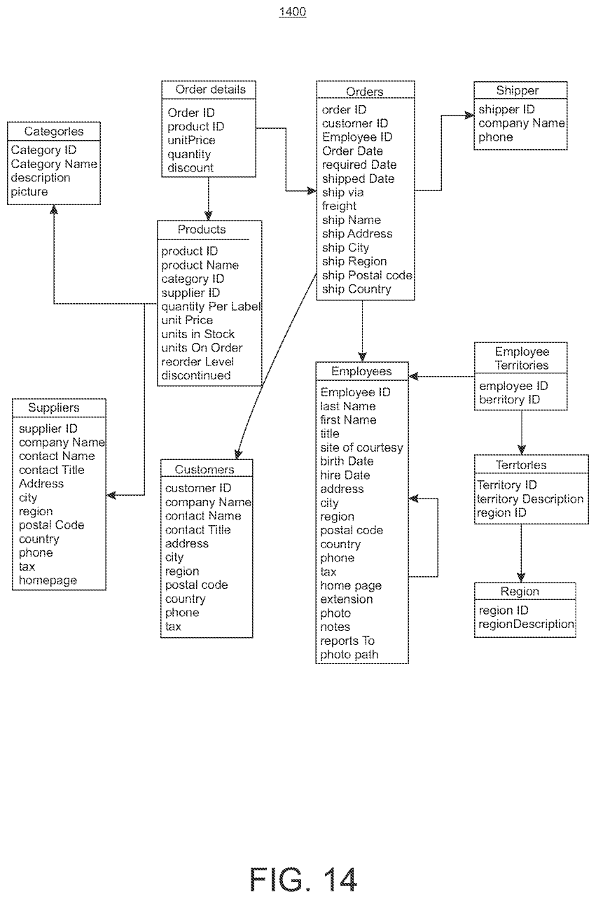

In further embodiments, attention may be applied recursively with multiple context vectors (also called nested attention). In this way, attention may be applied in system 100 to a collection of tables in a relational database. FIG. 14 is a diagram showing an example of eleven tables in a relational database (such as operational database 136) upon which nested attention is applied in a further embodiment of the present invention.

In one example as described above, a solicited procedure table (X) may be related to one other table in database 136 having historical procedures (H). The relationship between X and H can be due to a common key (e.g., a unique id of the patient soliciting the procedure) between X and H. For brevity, denote “related to” with a “->” and one obtains

X->H.

Each row r in X is related to variable number of (0, 1, or more rows) in H. That is, each patient in X that has a solicited procedure may have a variable number of historical procedures in H. Using the attention mechanism described herein with respect to attention-based neural network 120, for each row r in X, a) one may compute a fixed length context vector using related rows in H and b) concatenate the context vector to X's row. Do this for each row of X, one obtains a new table

X+C_H

where C_H is a table of fixed length context vectors, one for each row of X.

Consider when X is related to multiple tables in the database instead of just one. In this example, X may not only be related to H (as above) but may also be related to another table J. In this case, one obtains

X->H, X->J.

To handle this example, attention-based neural network 120 may compute two context vectors C separately. One context vector using attention on H. And another context vector using attention on J. Then the row r is concatenated with both the vectors. Doing so results in a new table

X+C_H+C_J.

In embodiments, attention-based neural network 120 may handle an arbitrary number of tables and relationships between them in a database 130. See FIG. 14 which shows 11 tables and the relationships among them. Applying attention-based neural network 120 and the systems and methods described herein to multiple tables and relationships is referred to as nested attention. For instance, consider when X is related to H and J; and H further is related to P and Q. One obtains

X->H, X->J, H->P, H->Q.

In this case, first, apply attention on H->P and H->Q and obtain H+C_P+C_Q. One can call this new table

M=H+C_P+C_Q.

Now, in this example one also has X->H and X->M. Applying attention on X->H and X->M, yields

X+C_H+C_M.

In this way, in embodiments, a recursive application of an attention mechanism (nested attention) can handle any number of database tables and relationships that can be represented with a Directed Acyclic Graph. For instance, a relational database 130 may include Directed Acyclic Graphs, and may allow self-edges. In case of a self edge for a table X, one obtains

X->X.

And one gets X+C_X, which is self attention. In the example of FIG. 14, there is a self edge for the Employees table.

### Column Weights

In a further embodiment, attention-based neural network 120 may learn column weights for feature selection. For example, using attention, column weights for feature selection may be learned along with row weights in historical procedures data H as described above.

As described above, attention-based neural network 120 computes weights for each row of H. Given X (solicited_procedures), with attention, design goals may be to find which historical procedures, to pay attention to, and how much attention systems and methods should pay for each historical procedure in H. In FIG. 7D, the row weights 750 are [0.20, 0.10, 0.40, . . . , 0.10] as described above with respect to FIG. 7C.

In a further feature, column weights (e.g, weighted averages of column data) may be computed as well for H (historic_procedures). As shown in the example data of FIG. 7D, column weights 780 are [0.01, . . . , 0.75]. While row weights 750 decide attention to rows of H (how much attention to each historical procedure), column weights 780 decide attention to columns of H. As columns of H are features of H, column weights 780 necessarily facilitate feature selection. Notice in FIG. 7D that feature x1 gets a column weight of 0.01 (lower importance), while feature x30 gets a column weight of 0.75 (higher importance). Just like the row weights 750 all add up to 1, column weights 780 should also all sum up to 1 in an embodiment.

Different ways to compute column weights with or without attention may be used in attention-based neural network 120. See, e.g., N. Gui et al., AFS: An Attention-based mechanism for Supervised Feature Selection, Proc. of AAAI Conference on Artificial Intelligence, vol. 33, no. 1, Jul. 17, 2019, pp. 3705-3713. In one embodiment, attention may be used to compute row weights. To compute row weights 750 for H, for example, one can use attention between X (solicited_procedures) and H. As described above, one may have Query=X, Key=H, Value=H, output from three separate Dense units 611-613. To be precise, Query=Dense_1(X), Key=Dense_2(H), and Value=Dense_3(H), where Dense_1, Dense_2, Dense_3 may be dense layers 611-613, respectively, or f, g, h respectively, as described above.

To compute column weights, attention-based neural network 120 can use attention with X and H_T, where H_T is the transpose of H. In other words, Query=Dense_1X, Key=Dense_2(H_T), Value=Dense_3(H_T). Attention-based neural network 120 may also be configured to consider self-attention with H_T (Query=Key=Value=H_T). Attention with X and H_T may find feature importance among columns/features of H given X. Self-attention with H_T may capture inter-feature correlations and dependencies if any between columns/features of H.

Attention with X, H, and H on row weights 750 results in a context vector, say C. Attention with X, H_T, and H_T will also result in another context vector, say C′, of the same shape. Attention-based neural network 120 can either concatenate the two context vectors C and C′ one after the other to X or concatenate a weighted (weights are to be learned) average of C and C′ to X to obtain a context vector 790 representative of attention involving row weights 750 and column weights 780.

FIG. 7D shows row weights 750 and column weights 780. Row weights 750 decide the importance (attention) of each procedure given X. Column weights 780 decide the importance (attention) of each feature given X. Note that such a depiction in FIG. 7D is only for clarity and simplicity. In reality, when C is computed as Attention(X, H, H) and C′ is computed as an Attention (X, H_T, H_T), the interplay of row weights and column weights (in other words, interplay of historical procedures in rows and features in columns) to determine an output context vector 790 may be even more complex than shown in FIG. 7D as would be apparent to a person skill in the art given this description.

### Further Example Computer-Implemented Implementations

Automated claim approval with machine learning as described herein can be implemented on or more computing devices. Computer-implemented functions and operations described above and with respect to embodiments shown in FIGS. 1-20 can be implemented in software, firmware, hardware or any combination thereof on one or more computing devices. In embodiments, system 100, including audit manager 110 and attention-based neural network 120, and processes 200-300 can be implemented in software, firmware, hardware or any combination thereof on one or more computing devices at the same or different locations.

Embodiments are also directed to computer program products comprising software stored on any computer-usable medium. Such software, when executed in one or more data processing devices, causes a data processing device(s) to operate as described herein or, as noted above, allows for the synthesis and/or manufacture of electronic devices (e.g., ASICs, or processors) to perform embodiments described herein. Embodiments employ any computer-usable or -readable medium, and any computer usable or -readable storage medium known now or in the future. Examples of computer usable or computer-readable mediums include, but are not limited to, primary storage devices (e.g., any type of random access memory), secondary storage devices (e.g., hard drives, floppy disks, CD ROMS, ZIP disks, tapes, magnetic storage devices, optical storage devices, MEMS, nano-technological storage devices, etc.), and communication mediums (e.g., wired and wireless communications networks, local area networks, wide area networks, intranets, etc.). Computer-usable or computer-readable mediums can include any form of transitory (which include signals) or non-transitory media (which exclude signals). Non-transitory media comprise, by way of non-limiting example, the aforementioned physical storage devices (e.g., primary and secondary storage devices).

The embodiments have been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific embodiments will so fully reveal the general nature of the embodiments that others can, by applying knowledge within the skill of the art, readily modify and/or adapt for various applications such specific embodiments, without undue experimentation, without departing from the general concept of the disclosure. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the embodiments should not be limited by any of the above-described exemplary embodiments, but should be defined only in accordance with the following claims and their equivalents.

What is claimed is:

1. A system for automated approval of claim requests for solicited procedures, comprising:
an audit manager;
an attention-based neural network coupled to the audit manager;
memory that stores tuning parameters and a set of risk level thresholds; and
a database configured to store training data, validation data and operation data,
wherein the training data includes fixed length data and variable length data, the fixed length data includes features and a target label, the variable length data including medical procedure code approval history data, and the operation data includes solicited procedures data and historical procedures data, and
wherein the audit manager is configured to output an approval indication for each solicited procedure according to a selected risk level threshold in the set of risk level thresholds.

2. The system of claim 1, wherein the attention-based neural network comprises an attention-based neural network trained according to the training data including the features and target label in the fixed length data and medical procedure code approval history data in the variable length data.

3. The system of claim 2, wherein the attention-based neural network is configured to output the tuning parameters corresponding to the trained attention-based neural network.

4. The system of claim 2, wherein the audit manager is configured to apply validation data to the trained attention-based neural network to determine the set of risk level thresholds.

5. The system of claim 2, wherein the audit manager is configured to, during an operation on a set of claim requests:
select a risk level threshold for a set of claim requests;
access solicited procedure data (X) for each claim request;
determine historical procedure data (H) associated with the accessed solicited procedure data;
feed the solicited procedure data (X) and determined historical procedure data (H) into the trained attention-based neural network to obtain a rejection probability score;
compare the obtained rejection probability score to the selected risk level threshold; and
output an approval indication for each claim request based on the comparison.

6. The system of claim 5, wherein the audit manager is configured to output the obtained rejection probability score for each claim request.

7. The system of claim 2, wherein the audit manager is configured to during training:
feed training data to the attention-based neural network, the training data including fixed length data including features and a target label and variable length data including medical procedure code approval history data;
receive a rejection probability score from the attention-based neural network;
determine an approval indication based on rejection probability score;
compare the determined approval indication with a target label in training data;
adjust tuning parameters of attention based neural network based on rejection probability scores and determined approval indication until training condition met; and
store set of tuning parameters in memory when training is complete.

8. The system of claim 7, wherein the attention-based neural network is configured to during training:
determine a fixed length context vector C based on the fixed length data and variable length data in the training data fed by the audit manager to the attention-based neural network;
generate fixed length attention data sequence A based on a concatenation of context vectors C and associated solicited procedure data;
feed generated fixed length attention data sequence A into a dense layer coupled to a sigmoid function unit to obtain a rejection probability score for output to the audit manager.

9. The system of claim 1, wherein the attention-based neural network includes a trained scalar dot-product attention neural network.

10. The system of claim 9, further comprising:
a concatenation unit coupled to an output of the trained scalar dot-product attention neural network;
a dense layer; and
a sigmoid function unit, wherein the dense layer is coupled to the output of the trained scalar dot-product attention neural network, and the sigmoid function unit is coupled to the output of the dense layer.

11. The system of claim 9, wherein the attention-based neural network is configured to apply one or more row weights, column weights, or a combination of row weights and column weights.

12. The system of claim 9, wherein the attention-based neural network is configured to apply attention or nested attention to one or more tables of data having one or more relationships between data in the tables of data.

13. The system of claim 1, wherein the audit manager is configured to provide output to an application on a remote computing device such that the remote application enables a remote user to view or select one or more display panels relating to a pre-audit of approved claim requests.

14. The system of claim 13, wherein:
at least one display panel indicates a level of risk for a solicited procedure, the level of risk determined based on a rejection probability score;
at least one display panel includes controls that allow a user to approve, reject or send a solicited procedure for further audit;
at least one display panel includes a control that allows a user to approve a group of solicited procedures having approval indications generated by the audit manager; or
at least one display panel includes a result dashboard having summary displays of results with respect to requests processed in a pre-audit having high, medium, and low risks.

15. A computer-implemented method for automated approval of claim requests for solicited procedures including fixed length and variable length data, comprising:
selecting a risk level threshold for a set of claim requests;
accessing solicited procedure data (X) for each claim request;
determining historical procedure data (H) associated with the accessed solicited procedure data;
feeding the solicited procedure data (X) and determined historical procedure data (H) into a trained attention-based neural network to obtain a rejection probability score;
comparing the obtained rejection probability score to the selected risk level threshold; and
outputting an approval indication for each claim request based on the comparison.

16. The method of claim 15, further including outputting the obtained rejection probability score for each claim request.

17. The method of claim 15, further comprising:
storing tuning parameters and a set of risk level thresholds in memory; and
storing training data, validation data and operation data in a database, wherein the training data includes fixed length data and variable length data, the fixed length data includes features and a target label, the variable length data including medical procedure code approval history data, and the operation data includes solicited procedures data and historical procedures data.

18. The method of claim 15, further comprising:
training an attention-based neural network according to the training data including the features and target label in the fixed length data and medical procedure code approval history data in the variable length data to obtain the trained attention-based neural network; and
outputting tuning parameters corresponding to the trained attention-based neural network.

19. The method of claim 18, further comprising applying validation data to the trained attention-based neural network to determine a set of risk level thresholds.

20. The method of claim 18, further comprising the steps of:
feeding training data to the attention-based neural network, the training data including fixed length data including features and a target label and variable length data including medical procedure code approval history data;
determining a rejection probability score;
determining an approval indication based on rejection probability score;
comparing the determined approval indication with a target label in training data;
adjusting tuning parameters of attention based neural network based on the rejection probability scores and determined approval indication until a training condition met; and
storing a set of tuning parameters in memory when training is complete.

21. The method of claim 20, wherein the training attention-based neural network step includes the steps of:
determining a fixed length context vector C based on the fixed length data and variable length data in the training data fed to the attention-based neural network;
generating fixed length attention data sequence A based on a concatenation of context vectors C and associated solicited procedure data;
feeding generated fixed length attention data sequence A into a dense layer coupled to a sigmoid function unit to obtain a rejection probability score.

22. The method of claim 20, wherein the feeding training data to the attention-based neural network includes the steps of:
inputting X into first dense layer to obtain queries Q;
inputting H into second dense layer to obtain keys K;
inputting H into third dense layer to obtain values V;
computing a scaled dot product between all pairs of queries Q and keys K;
masking irrelevant weights;
normalizing masked weights; and
computing weighted averages of historical procedure values V.

23. A non-transitory computer-readable medium for automating approval of claim requests for solicited procedures including fixed length and variable length data, the medium having instructions stored thereon, that when executed by at least one processor, cause the at least one processor to:
select a risk level threshold for a set of claim requests;
access solicited procedure data (X) for each claim request;
determine historical procedure data (H) associated with the accessed solicited procedure data;
feed the solicited procedure data (X) and determined historical procedure data (H) into a trained attention-based neural network to obtain a rejection probability score;
compare the obtained rejection probability score to the selected risk level threshold; and
output an approval indication for each claim request based on the comparison.

24. The medium of claim 23, wherein the trained attention-based neural network comprises a trained scalar dot-product attention neural network.
